(19) United States
(12) Patent Application Publication
(10) Pub. No.: US 2023/0398322 A1
(43) Pub. Date: Dec. 14, 2023

(54) AUTOMATIC OXYGEN TITRATION SYSTEM AND METHOD BASED ON TARGETED OXYGEN SATURATION DURING HIGH FLOW AND LOW FLOW OXYGEN THERAPY

(71) Applicant: Rush University Medical Center, Chicago, IL (US)

(72) Inventors: Ahmad Aouni ELSHAFEI, Darien, IL (US); Ahmad Issam EL SHAFEI, Glendale, WI (US)

(73) Assignee: Rush University Medical Center, Chicago, IL (US)

(21) Appl. No.: 18/209,867

(22) Filed: Jun. 14, 2023

Related U.S. Application Data
(60) Provisional application No. 63/351,966, filed on Jun. 14, 2022.

(51) Int. Cl. A61M 16/10; A61B 5/1455

(52) U.S. Cl. CPC A61M 16/1005; A61B 5/14552; A61M 2016/1025

(57) ABSTRACT

This disclosure provides for automatic oxygen titration in high flow and low flow oxygen therapy based on targeted oxygen saturation levels. For example, this disclosure involves an algorithm that can automatically titrate and adjust oxygen fraction (FiO₂) of an oxygen therapy medical device based on the peripheral capillary oxygen saturation (SpO₂) of the patient to achieve a targeted SpO₂. This can allow for continuously monitoring and recording of delivered FiO₂ and SpO₂. In addition, the administered FiO₂ can be adjusted according to patient SpO₂ and target SpO₂ goals. The disclosure can be adapted to any high flow oxygen device and any low flow oxygen device, such as a flowmeter. In addition, clinicians or other users can be alerted about changes in a subject's oxygenation status with an alarm function.

### CROSS-REFERENCE TO RELATED APPLICATIONS

This application claims the benefit of U.S. Provisional Application No. 63/351,966, entitled “AUTOMATIC OXYGEN TITRATION SYSTEM AND METHOD BASED ON TARGETED OXYGEN SATURATION DURING HIGH FLOW AND LOW FLOW OXYGEN THERAPY,” filed on Jun. 14, 2022. The entire contents of this application are hereby incorporated by reference in their entirety.

### FIELD

This disclosure relates generally to oxygen therapy and, more particularly, to systems and methods for adjusting oxygen delivery based on target oxygen saturation.

### BACKGROUND

Oxygen therapy involves the delivery of supplemental oxygen to patients with lower oxygen levels in need of respiratory help. A nasal cannula is a device commonly used to deliver oxygen therapy and includes a tube with prongs or outlets placed into a patient's nostrils to deliver a mixture of air and oxygen from a source. Two common types of nasal cannulas utilized to deliver supplemental oxygen include low flow and high flow nasal cannulas.

A low flow nasal cannula may deliver around 1-6 liters of oxygen per minute, with even smaller flow rates possible for cannulas intended for use with infants, for example. High Flow Nasal Cannula (HFNC) is a common modality of oxygen therapy to treat hypoxemia patients. The benefit of HFNC is that it delivers gas flow and oxygen to the patient, and can do so at rates much higher than low flow nasal cannulas, e.g., up to 60 liters of oxygen per minute. In addition, the gas flow delivered to the patient can be heated to body temperature and humidified to prevent drying out the airway, which can lead to inflammation.

Most HFNC devices allow for setting an appropriate oxygen fraction (FiO₂). FiO₂ is titrated by clinicians as appropriate to achieve a targeted peripheral capillary oxygen saturation (SpO₂). Proper titration can involve significant interaction between clinicians and patients, however, and involve significant clinician time to monitor and adjust the flow properly. In addition to the cost associated with clinician time monitoring the therapy, there can be an increased risk of nosocomial infection for a patient from the increased interactions with the clinician.

Accordingly, there is a need for improved systems and methods for oxygen therapy, including such systems and methods with an ability to automatically adjust therapy parameters during high flow and low flow oxygen therapy.

### SUMMARY

This disclosure provides various devices, systems, and methods for automatic oxygen titration in high flow and low flow oxygen therapy. The disclosed devices, systems, and methods can perform automatic oxygen titration based on targeted oxygen saturation levels. For example, this disclosure involves an algorithm that can automatically titrate and adjust oxygen fraction (FiO₂) of an oxygen therapy medical device based on the peripheral capillary oxygen saturation (SpO₂) of the patient to achieve a targeted SpO₂. The system and method can allow for continuously monitoring and recording of delivered FiO₂ and SpO₂. In addition, the system and method can allow for adjusting the administered FiO₂ according to patient SpO₂ and setting target SpO₂ goals. The system and method can be adaptable to any high flow oxygen device and adaptable to any low flow oxygen device, such as a flowmeter. The system and method can also alert clinicians about changes in patient's oxygenation status with an alarm function.

In one aspect, a method for oxygen titration for a subject is disclosed that includes receiving a target peripheral capillary oxygen saturation (SpO₂) range for a subject, as well as sensing a subject's SpO₂ level. The method further includes comparing the subject's SpO₂ level to the target SpO₂ range for the subject using a controller, and actuating a valve coupled to an oxygen (O₂) reservoir to modulate flow of O₂ when the subject's SpO₂ level is outside of the target SpO₂ range to deliver O₂ to the subject.

Any of a variety of alternative or additional features can be included and are considered within the scope of the present disclosure. For example, in some embodiments, sensing a subject's SpO₂ level can be performed using a pulse oximeter coupled to the subject.

In certain embodiments, receiving the target SpO₂ range can include detecting input from a user via an interface coupled to the controller.

In some embodiments, delivering O₂ to the subject can include flowing O₂ through a high flow nasal cannula. In some embodiments, the flow rate of O₂ can be between about 7 liters per minute and about 60 liters per minute. In some embodiments, the flow rate of O₂ can be between about 20 liters per minute and about 60 liters per minute.

In certain embodiments, delivering O₂ to the subject can include flowing O₂ through a low flow nasal cannula. In certain embodiments, the flow rate of O₂ can be between about 1 liter per minute and about 6 liters per minute.

In some embodiments, the method can further include measuring O₂ concentration prior to delivery to the subject. In some embodiments, the method can further include communicating measured O₂ concentration to the controller for use in modulating flow of O₂. In some embodiments, the method can further include notifying a user if measured O₂ concentration falls below a threshold value. In some embodiments, notifying the user can include actuating any of a visual, haptic, or audio alarm. In some embodiments, notifying the user can include interacting with a remotely disposed device through a communications network.

In certain embodiments, the method can further include notifying a user if the sensed SpO₂ level falls below a threshold value. In certain embodiments, notifying the user can include actuating any of a visual, haptic, or audio alarm. In certain embodiments, notifying the user can include interacting with a remotely disposed device through a communications network.

In another aspect, a system for oxygen titration for a subject is disclosed that includes a pulse oximeter coupled to a subject, an oxygen (O₂) reservoir, a valve coupled to the O₂ reservoir, and an O₂ delivery device coupled to the O₂ reservoir and configured to deliver O₂ to the subject. The system further includes a controller configured to receive a target peripheral capillary oxygen saturation (SpO₂) range for the subject, receive a peripheral capillary oxygen saturation (SpO₂) for the subject from the pulse oximeter, and actuate the valve to modulate flow of O₂ from the O₂ reservoir to the O₂ delivery device when the subject's SpO₂ level is outside of the target SpO₂ range to deliver O₂ to the subject.

As with the methods described above, the systems disclosed herein can include any of a variety of additional or alternative features that are considered within the scope of the present disclosure. For example, in some embodiments the system can further include an O₂ concentration sensor configured to measure a concentration of oxygen flowing through the O₂ delivery device.

In certain embodiments, the system can further include an alarm configured to produce any of a visual, haptic, or audio notification.

In some embodiments, the system can further include an interface for receiving input from a user.

In certain embodiments, the O₂ delivery device can be a high flow nasal cannula. In certain embodiments, the system can further include a humidifier.

In some embodiments, the O₂ delivery device can be a low flow nasal cannula.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a schematic illustration of one embodiment of a system according to the present disclosure; and

FIG. 2 is a flow chart illustrating one embodiment of a method according to the present disclosure.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

As noted above, this disclosure provides various devices, systems, and methods for automatic oxygen titration in high flow and low flow oxygen therapy. The disclosed devices, systems, and methods can perform automatic oxygen titration based on targeted oxygen saturation levels. For example, this disclosure involves an algorithm that can automatically titrate and adjust oxygen fraction (FiO₂) of an oxygen therapy medical device based on the peripheral capillary oxygen saturation (SpO₂) of the patient to achieve a targeted SpO₂. The system and method can allow for continuously monitoring and recording of delivered FiO₂ and SpO₂. In addition, the system and method can allow for adjusting the administered FiO₂ according to patient SpO₂ and setting target SpO₂ goals. The system and method can be adaptable to any high flow oxygen device and adaptable to any low flow oxygen device, such as a flowmeter. The system and method can also alert clinicians about changes in patient's oxygenation status with an alarm function.

The disclosed devices, systems, and methods can provide a number of advantages over conventional oxygen delivery. For example, proper titration can involve significant interaction between clinicians and patients, as well as significant clinician time to monitor and adjust the flow properly. In addition to the cost associated with clinician time monitoring the therapy, there can be an increased risk of nosocomial infection for a patient from the increased interactions with the clinician. The disclosed devices, systems, and methods can provide for automatic oxygen titration during high flow and low flow oxygen therapy, which can reduce the amount of time needed for a clinician or other user to administer the therapy. This can have direct benefits, such as reduced time and associated cost to administer the therapy, as well as ancillary benefits, such as a reduced risk for nosocomial infection due to less interaction between clinicians and patients.

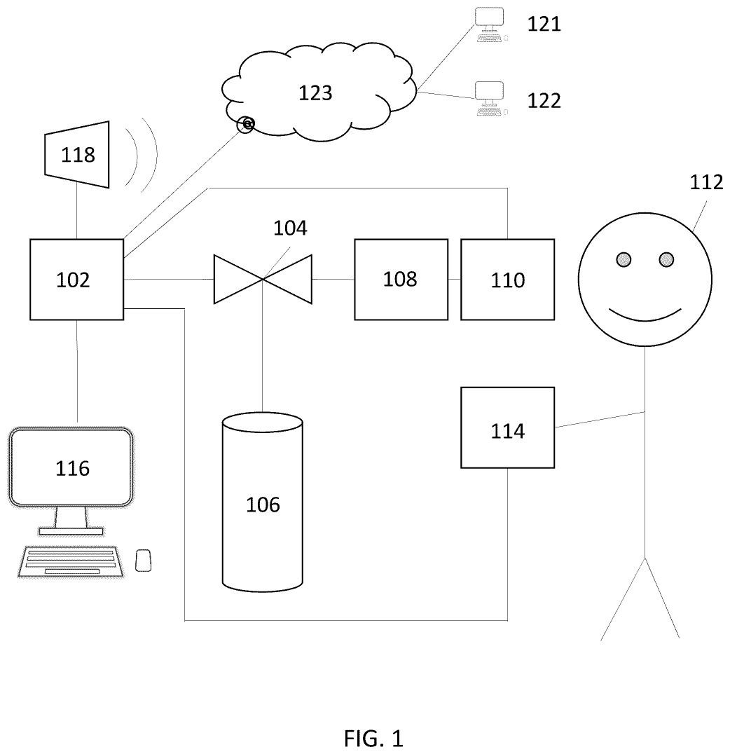

FIG. 1 illustrates one embodiment of a system 100 according to the present disclosure. The system 100 can include a controller 102, such as a Field Programmable Gate Array (FPGA) chip or other electronic component or assembly, configured to receive inputs from various system components and control other system components based on said inputs.

The system can also include a valve 104, such as an electronically adjustable servo-controlled valve, that can control a volume of oxygen (O₂) gas flowing from an O₂ supply, such as a reservoir, generator, or other source.

The system can further include an O₂ delivery device 108, such as a low flow nasal cannula or a high flow nasal cannula. For example, in some embodiments, a low flow nasal cannula configured to deliver between about 1 liter per minute and about 6 liters per minute to a patient can be utilized. In other embodiments, a high flow nasal cannula configured to deliver between about 7 liters per minute and about 60 liters per minute to a patient can be utilized. In certain embodiments, the high flow nasal cannula can be configured to deliver between about 20 liters per minute and about 60 liters per minute to a patient. In some embodiments, a high flow nasal cannula can include a humidifier and/or heating element to provide warm and high humidity O₂ gas flow.

The system can further include an O₂ analyzer or concentration sensor 110 that can be configured to detect a concentration of O₂ in the gas flow being delivered to a patient 112 through the O₂ delivery device 108. The O₂ analyzer can be coupled to the controller 102 such that data detected by the O₂ analyzer can be communicated back to the controller for use in controlling the therapy.

The system can also include a pulse oximeter 114 to measure a patient's peripheral capillary oxygen saturation (SpO₂). The pulse oximeter 114 can be coupled to the controller 102 such that data detected by the pulse oximeter can be communicated back to the controller for use in controlling the therapy.

The system can further include an interface 116 coupled to the controller 102 and configured to allow a user to input data to the controller. For example, a display with keyboard and/or mouse can be provided as an interface to enable communication between a user and the controller 102. Any of a variety of alternative interfaces can also be utilized, including, for example, various button controls, touchscreen controls, voice controls, virtual reality (VR) or augmented reality (AR) interfaces, etc.

The system can also include an alarm 118 configured to notify a user via any of audio, visual, haptic, or other user-detectable communications. Still further, the controller 102 can be coupled to one or more additional computing devices, e.g., devices 121, 122, via a communications network 123, such as the internet.

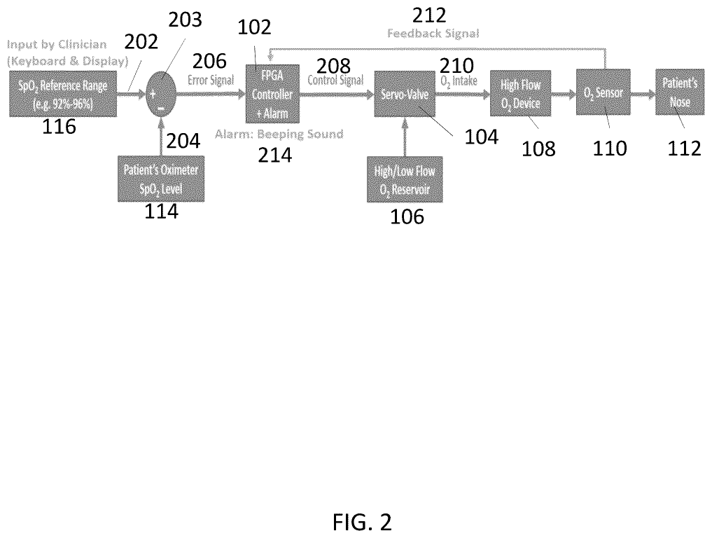

Below is a description of certain operations and features of the systems and methods disclosed herein. For example, and with reference to the flow diagram of FIG. 2 in combination with the schematic system of FIG. 1, a subject or patient's SpO₂ can be detected using the pulse oximeter 114 and communicated to the field-programmable gate array (FPGA) controller 102, as shown by signal 204. This communication, and any communication described herein between various components of the system, can be performed via a wired or wireless connection between the components with the possibility of one or more intervening components disposed therebetween (e.g., a network router, etc.).

In addition, a clinician or other user can enter a desired SpO₂ target range via the interface 116, as shown by signal 202. As shown at operation 203, the detected SpO₂ of the patient can be compared to the desired target range and a resulting error signal 206 can be received by the controller 102 as a representation of whether the detected SpO₂ is within the desired target range or not.

The controller 102 can then control an actuator and/or valve 104 via signal 208 and titrate FiO₂ connected to the high-flow oxygen device 108 in-order to achieve the desired SpO₂ targets in the patient. This automatic titration does not require physical presence of clinicians and will speed up the intervention, as well as enhance patient outcomes. The system can also include an alarm function, e.g., when the FiO₂ meets a preset alarm criteria (e.g., 20% above the current FiO₂), the system can alarm clinicians to check on the patient in-order to ensure patient's safety.

A high-performing OEM pulse oximeter 114 can be utilized to sense the patient's SpO₂ level. Data can be continuously sampled and transmitted to the controller 102.

The controller 102 can continuously compare the patient's SpO₂ level with a defined range of SpO₂ (e.g., 92%-96%), which can be controlled and pre-set by the clinician.

The error signal 206 (e.g., defined as the clinician-defined range of SpO₂ minus the detected patient SpO₂ level) can be sent to the servo valve 104, which can be connected to the O₂ reservoir. In some embodiments, the magnitude of the error signal can control the degree to which the valve is opened and O₂ is allowed to flow from the O₂ reservoir.

If the error signal falls below a clinician “ALARM” level or predetermined threshold value (e.g., a drop of 20% in patient's SpO₂ level), an alarm beeping sound 214 can be activated to notify the clinician to take immediate necessary medical action. In some embodiments, alternatively or additionally, the controller 102 can communicate with a remote computing device that is with the clinician (e.g., a mobile phone, etc.), to provide the alarm warning, as well as specific data on the nature of the alarm and detected values that triggered it.

As noted above, the magnitude of the error signal 206 can be utilized to control the valve 104. In some embodiments, based on the error signal 206, the FPGA controller 102 can send a Pulse Width Modulated (PWM) signal 208 to open the servo valve 104 as much as needed.

Servo valve control can be based on O₂ flow calculation, which relates the flow cubic meter with the O₂ reservoir liters capacity.

The valve 104 can be configured to handle low flow and high flow levels applicable to clinical settings, such as a hospital, as well as home care applications.

The servo valve output 210 can be coupled to an oxygen delivery device 108. The oxygen delivery device 108 can be a high flow high humidity device in some embodiments. That is, the device 108 can include a humidifier with an integrated flow generator that delivers high flow warmed and humidified respiratory gases to, e.g., spontaneously breathing patients through a variety of patient interfaces.

For visual display, redundancy, and system reliability, the output of the oxygen delivery device 108 can be coupled to an oxygen analyzer or sensor 110, which can measure and display the oxygen concentration in a flow of gas from a medical gas-flow device. The oxygen sensor 110 can send a feedback signal 212 to the controller 102 so that it can validate the accurate operation of the servo-valve 104.

The above-described control loop can cycle continuously to provide automatic titration of oxygen during administration of high flow or low flow oxygen therapy to a patient or subject. As noted above, FIG. 2 illustrates an embodiment of the control algorithm.

Trend data of patient SpO₂ and delivered FiO₂ can be recorded for later retrieval and/or analysis. Data can be sent via Bluetooth, internet, or other communication connection to authorized personnel and/or central monitoring systems. Any alarms or other notifications can also be sent via Bluetooth, internet, or other communication connection to authorized personnel.

The system and method described can be applicable to any of current commercially available low flow and high flow nasal gas devices, which are broadly utilized inside and outside of clinical settings, such as hospitals, etc.

In acute and critical care environments, the disclosed systems can allow a more efficient titration of oxygen fraction (FiO₂) in accordance with patient needs and targeted SpO₂ (peripheral capillary oxygen saturation) to better meet therapeutic oxygenation goals. This is provided along with the advantages of monitoring changes in oxygenation in real time and providing for alarms in the event of undesirable events.

The system and method can be utilized in a variety of contexts, including, for example, for patients with chronic pulmonary disease in a home setting, allowing for improved monitoring of oxygen settings while maintaining close monitoring of patient status by authorized personnel.

High Flow Nasal Cavity (HFNC) has been utilized broadly worldwide due to its benefits on oxygenation and humidification. It benefits a wide range of patients, from premature infants to the elderly who have respiratory compromise or who need better humidification. The systems and methods described herein can be equipped on any of current HFNC device or can be built separately. Additionally, the systems can be added to low flow oxygen flow meters to allow for automatic flow titration.

In this disclosure, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A method for oxygen titration for a subject, the method comprising:
   receiving a target peripheral capillary oxygen saturation (SpO₂) range for the subject;
   sensing a subject's SpO₂ level;
   comparing the subject's SpO₂ level to the target SpO₂ range for the subject using a controller; and
   actuating a valve coupled to an oxygen (O₂) reservoir to modulate flow of O₂ when the subject's SpO₂ level is outside of the target SpO₂ range to deliver O₂ to the subject.
2. The method of claim 1, wherein sensing a subject's SpO₂ level is performed using a pulse oximeter coupled to the subject.
3. The method of any of claims 1 to 2, wherein receiving the target SpO₂ range includes detecting input from a user via an interface coupled to the controller.
4. The method of any of claims 1 to 3, wherein delivering O₂ to the subject includes flowing O₂ through a high flow nasal cannula.
5. The method of claim 4, wherein the flow rate of O₂ is between about 7 liters per minute and about 60 liters per minute.
6. The method of any of claims 1 to 3, wherein delivering O₂ to the subject includes flowing O₂ through a low flow nasal cannula.
7. The method of claim 6, wherein the flow rate of O₂ is between about 1 liter per minute and about 6 liters per minute.
8. The method of any of claims 1 to 7, further comprising measuring O₂ concentration prior to delivery to the subject.
9. The method of claim 8, further comprising communicating measured O₂ concentration to the controller for use in modulating flow of O₂.
10. The method of any of claims 8 to 9, further comprising notifying a user if measured O₂ concentration falls below a threshold value.
11. The method of claim 10, wherein notifying the user includes actuating any of a visual, haptic, or audio alarm.
12. The method of any of claims 10 to 11, wherein notifying the user includes interacting with a remotely disposed device through a communications network.
13. The method of any of claims 1 to 12, further comprising notifying a user if the sensed SpO₂ level falls below a threshold value.
14. The method of claim 13, wherein notifying the user includes actuating any of a visual, haptic, or audio alarm.
15. The method of any of claims 13 to 14, wherein notifying the user includes interacting with a remotely disposed device through a communications network.
16. A system for oxygen titration for a subject, the system comprising:
   a pulse oximeter coupled to a subject;
   an oxygen (O₂) reservoir;
   a valve coupled to the O₂ reservoir;
   an O₂ delivery device coupled to the O₂ reservoir and configured to deliver O₂ to the subject; and
   a controller configured to:
      receive a target peripheral capillary oxygen saturation (SpO₂) range for the subject;
      receive a peripheral capillary oxygen saturation (SpO₂) for the subject from the pulse oximeter;
      actuate the valve to modulate flow of O₂ from the O₂ reservoir to the O₂ delivery device when the subject's SpO₂ level is outside of the target SpO₂ range to deliver O₂ to the subject.
17. The system of claim 16, further comprising an O₂ concentration sensor configured to measure a concentration of oxygen flowing through the O₂ delivery device.
18. The system of any of claims 16 to 17, further comprising an alarm configured to produce any of a visual, haptic, or audio notification.
19. The system of any of claims 16 to 18, further comprising an interface for receiving input from a user.
20. The system of any of claims 16 to 19, wherein the O₂ delivery device is a high flow nasal cannula.
21. The system of claim 20, further comprising a humidifier.
22. The system of any of claims 16 to 19, wherein the O₂ delivery device is a low flow nasal cannula

What is claimed is:

1. A method for oxygen titration for a subject, the method comprising:
receiving a target peripheral capillary oxygen saturation (SpO₂) range for the subject;
sensing a subject's SpO₂ level;
comparing the subject's SpO₂ level to the target SpO₂ range for the subject using a controller; and
actuating a valve coupled to an oxygen (O₂) reservoir to modulate flow of O₂ when the subject's SpO₂ level is outside of the target SpO₂ range to deliver O₂ to the subject.

2. The method of claim 1, wherein sensing a subject's SpO₂ level is performed using a pulse oximeter coupled to the subject.

3. The method of claim 1, wherein receiving the target SpO₂ range includes detecting input from a user via an interface coupled to the controller.

4. The method of claim 1, wherein delivering O₂ to the subject includes flowing O₂ through a high flow nasal cannula.

5. The method of claim 4, wherein the flow rate of O₂ is between about 7 liters per minute and about 60 liters per minute.

6. The method of claim 1, wherein delivering O₂ to the subject includes flowing O₂ through a low flow nasal cannula.

7. The method of claim 6, wherein the flow rate of O₂ is between about 1 liter per minute and about 6 liters per minute.

8. The method of claim 1, further comprising measuring O₂ concentration prior to delivery to the subject.

9. The method of claim 8, further comprising communicating measured O₂ concentration to the controller for use in modulating flow of O₂.

10. The method of claim 8, further comprising notifying a user if measured O₂ concentration falls below a threshold value.

11. The method of claim 10, wherein notifying the user includes actuating any of a visual, haptic, or audio alarm.

12. The method of claim 10, wherein notifying the user includes interacting with a remotely disposed device through a communications network.

13. The method of claim 1, further comprising notifying a user if the sensed SpO₂ level falls below a threshold value.

14. The method of claim 13, wherein notifying the user includes actuating any of a visual, haptic, or audio alarm.

15. The method of claim 13, wherein notifying the user includes interacting with a remotely disposed device through a communications network.

16. A system for oxygen titration for a subject, the system comprising:
a pulse oximeter coupled to a subject;
an oxygen (O₂) reservoir;
a valve coupled to the O₂ reservoir;
an O₂ delivery device coupled to the O₂ reservoir and configured to deliver O₂ to the subject; and
a controller configured to:
receive a target peripheral capillary oxygen saturation (SpO₂) range for the subject;
receive a peripheral capillary oxygen saturation (SpO₂) for the subject from the pulse oximeter;
actuate the valve to modulate flow of O₂ from the O₂ reservoir to the O₂ delivery device when the subject's SpO₂ level is outside of the target SpO₂ range to deliver O₂ to the subject.

17. The system of claim 16, further comprising an O₂ concentration sensor configured to measure a concentration of oxygen flowing through the O₂ delivery device.

18. The system of claim 16, further comprising an alarm configured to produce any of a visual, haptic, or audio notification.

19. The system of claim 16, further comprising an interface for receiving input from a user.

20. The system of claim 16, wherein the O₂ delivery device is a high flow nasal cannula.

21. The system of claim 20, further comprising a humidifier.

22. The system of claim 16, wherein the O₂ delivery device is a low flow nasal cannula.
